(19) United States
(12) Patent Application Publication
(10) Pub. No.: US 2020/0107791 A1
(43) Pub. Date: Apr. 9, 2020

(54) RADIOGRAPHIC IMAGING APPARATUS AND STORAGE MEDIUM

(71) Applicant: KONICA MINOLTA, INC., Tokyo (JP)

(72) Inventor: Toi SAWAGUCHI, Tokyo (JP)

(21) Appl. No.: 16/589,426

(22) Filed: Oct. 1, 2019

(30) Foreign Application Priority Data
Oct. 3, 2018 (JP) 2018-188386

(51) Int. Cl. A61B 6/00

(52) U.S. Cl. CPC A61B 6/4266; H01L 27/14663; A61B 6/4233

(57) ABSTRACT

A radiographic imaging apparatus includes: a hardware processor; and a correlated double sampling circuit including first and second sample-and-hold circuits. The hardware processor causes the first sample-and-hold circuit to perform sampling for a predetermined period of time; causes the second sample-and-hold circuit to perform sampling for a period of time longer than time constant of the second sample-and-hold circuit; obtains correction information based on a difference output from the correlated double sampling circuit every predetermined number of times the sampling in a non-conductive state is performed; calculates a correction value based on the obtained correction information; corrects, with the calculated correction value, a signal value that is the difference output from the correlated double sampling circuit after the second sample-and-hold circuit performs the sampling via a conductive state; and generates a radiograph based on the corrected signal value.

### CROSS REFERENCE TO RELATED APPLICATION

The present invention claims priority under 35 U.S.C. § 119 to Japanese Patent Application No. 2018-188386 filed Oct. 3, 2018, the entire content of which is incorporated herein by reference.

### BACKGROUND

### Technological Field

The present disclosure relates to a radiographic imaging apparatus and a storage medium.

### Description of the Related Art

In a radiographic imaging apparatus that accumulates charges in amounts corresponding to doses of received radiation in radiation detection elements, reads the amounts of accumulated charges as analog signal values, and digitalizes the analog signal values, thereby generating image data of radiographs, the amounts of charges accumulated in the respective radiation detection elements are very small and difficult to digitalize accurately if nothing is done. Hence, readout circuits that read signal values need to have amplifier circuits that amplify current flowing in from the radiation detection elements. However, such a configuration makes noise enter signal values when the amplifier circuits amplify the signal values (current).

Then, there is a conventional technology of repeatedly reading the amounts of charges (signal values) while TFTs (Thin Film Transistors) connected to scan lines are in OFF state (hereinafter “TFT-OFF readout”), calculating the average value(s) of the obtained signal values as offset data, and correcting a radiograph by subtracting the offset data from image data of a taken image read while the TFTs are in a conductive state. (See JP 2001-141832 A.)

In recent years, there have been developed radiographic imaging apparatuses for video shooting, in which charges are accumulated and read repeatedly at high speed.

In video shooting, charges are accumulated and read repeatedly for a relatively long period of time (e.g. about tens of seconds). Consequently, during video shooting, temperature of readout circuits gradually increases and responsiveness thereof decreases accordingly, so that a phenomenon called offset drift, where signal values to be read change with time, occurs.

Then, there has been a technology of: performing TFT-OFF readout every time a frame of a video is generated, thereby obtaining offset data for each frame; calculating a drift amount of offset on the basis of each offset data and reference data obtained beforehand; and correcting each frame by subtracting its corresponding offset data and drift amount from the frame. (See US 2017/0278277 A1)

In order to remove noise that enters signal values at the time of amplifying the signal values, correlated double sampling (CDS) circuits are used in general.

The CDS circuits each have two capacitors, and charge one capacitor with an output voltage at the time of TFT-OFF readout (sampling) and hold a voltage between the electrodes of the capacitor as a first voltage (sample-holding), and charge the other capacitor with an output voltage at the time of readout/reading with the TFTs put in the conductive state after radiation irradiation and hold a voltage between the electrodes of the capacitor as a second voltage. Then, the CDS circuits each output a signal value with a noise component removed by subtracting the first voltage from the second voltage. (See FIG. 3, for example.)

The abovementioned offset drift is mainly caused by the following factor: because sample-and-hold circuits of each CDS circuit, which form a pair, are different from one another in degree of decrease of responsiveness, a gap in responsiveness between the sample-and-hold circuits appears and becomes bigger with time. (See FIG. 7A and FIG. 7B.)

To deal with this, it is required to sample the first voltage(s) and the second voltage(s) accurately.

By the way, it takes some time (resistor-capacitor (RC) time constant τ or more of a CDS circuit) for a voltage to be converged since a capacitor starts being charged.

Furthermore, it is known that through current flows between pixels and amplifier circuits when images are read with TFTs put in the conductive state. Through current is generated when TFTs are turned on or off, by charges going back and forth between (i) a parasitic capacitance (“C” in FIG. 8) that is present between the gate and the source of each TFT and (ii) a capacitor of each amplifier circuit (integrating circuit). (See FIG. 8, for example.) Due to this through current, output voltages of the amplifier circuits temporarily decrease when the TFTs are put in the conductive state.

This phenomenon is no problem if charging time of each capacitor is long enough. However, in the case of video shooting performed at a high frame rate (e.g. 15 frames per second), reading time for each row is short, and hence sampling time for the second voltages is shorter than the RC time constant i of the CDS circuits. As a result, the sampling is performed in a state in which the temporarily-decreased output voltages of the amplifier circuits are not converged. This causes a problem that accurate second voltages cannot be obtained.

In addition, if temperature of the CDS circuits increases and responsiveness thereof decreases, the output voltages are further unlikely to be converged.

As a result, the drift amounts of offset at the time of TFT-OFF readout and the drift amounts of offset at the time of image readout/reading do not agree, and hence correction values for correcting images cannot be generated. No consideration is given to this kind of problem in the conventional technologies disclosed in JP 2001-141832 and US 2017/0278277.

### SUMMARY

Objects of the present disclosure include increasing accuracy of offset correction in video shooting in a radiographic imaging apparatus including: radiation detection elements that generate charges in amounts corresponding to doses of received radiation; integrating circuit(s) that output voltages obtained by time integration of the amounts of flowed-in charges; and switch elements that switch a conductive state in which the radiation detection elements and the integrating circuits are electrically conductive to one another and a non-conductive state in which the radiation detection elements and the integrating circuits are not electrically conductive to one another.

In order to achieve at least one of the abovementioned objects, according to a first aspect of the present invention, there is provided a radiographic imaging apparatus including:

a plurality of radiation detection elements that are arranged so as to spread two-dimensionally and generate charges in amounts corresponding to doses of received radiation;

an integrating circuit that outputs a voltage obtained by time integration of an amount of flowed-in charges;

a correlated double sampling circuit that: has (i) a first sample-and-hold circuit that samples the voltage output from the integrating circuit and holds the sampled voltage as a first voltage and (ii) a second sample-and-hold circuit that samples the voltage output from the integrating circuit and holds the sampled voltage as a second voltage; and outputs a difference between the second voltage and the first voltage;

a plurality of switch elements that switch a conductive state in which the corresponding radiation detection elements and the integrating circuit are electrically conductive to one another and a non-conductive state in which the corresponding radiation detection elements and the integrating circuit are not electrically conductive to one another; and

a hardware processor that:
causes the first sample-and-hold circuit to sample the voltage output from the integrating circuit for a predetermined period of time every time the integrating circuit finishes resetting a voltage;
causes the second sample-and-hold circuit to sample the voltage output from the integrating circuit for a period of time longer than time constant of the second sample-and-hold circuit after the first sample-and-hold circuit finishes the sampling;
obtains correction information based on the difference output from the correlated double sampling circuit every predetermined number of times the sampling in the non-conductive state is performed by the first sample-and-hold circuit and the second sample-and-hold circuit;
calculates a correction value based on the obtained correction information; corrects, with the calculated correction value, a signal value that is the difference output from the correlated double sampling circuit after the second sample-and-hold circuit performs the sampling via the conductive state; and
generates a radiograph based on the corrected signal value.

According to a second aspect of the present invention, there is provided a non-transitory computer-readable storage medium storing a program to cause, of a radiographic imaging apparatus including: a plurality of radiation detection elements that are arranged so as to spread two-dimensionally and generate charges in amounts corresponding to doses of received radiation; an integrating circuit that outputs a voltage obtained by time integration of an amount of flowed-in charges; a correlated double sampling circuit that: has (i) a first sample-and-hold circuit that samples the voltage output from the integrating circuit and holds the sampled voltage as a first voltage and (ii) a second sample-and-hold circuit that samples the voltage output from the integrating circuit and holds the sampled voltage as a second voltage; and outputs a difference between the second voltage and the first voltage; and a plurality of switch elements that switch a conductive state in which the corresponding radiation detection elements and the integrating circuit are electrically conductive to one another and a non-conductive state in which the corresponding radiation detection elements and the integrating circuit are not electrically conductive to one another, a computer to:

cause the first sample-and-hold circuit to sample the voltage output from the integrating circuit for a predetermined period of time every time the integrating circuit finishes resetting a voltage;

cause the second sample-and-hold circuit to sample the voltage output from the integrating circuit for a period of time longer than time constant of the second sample-and-hold circuit after the first sample-and-hold circuit finishes the sampling;

obtain correction information based on the difference output from the correlated double sampling circuit every predetermined number of times the sampling in the non-conductive state is performed by the first sample-and-hold circuit and the second sample-and-hold circuit;

calculate a correction value based on the obtained correction information;

correct, with the calculated correction value, a signal value that is the difference output from the correlated double sampling circuit after the second sample-and-hold circuit performs the sampling via the conductive state; and

generate a radiograph based on the corrected signal value.

### BRIEF DESCRIPTION OF THE DRAWINGS

The objects, advantages, and features provided by one or more embodiments of the present invention will become more fully understood from the detailed description given hereinbelow and the appended drawings that are given by way of illustration only, and thus are not intended as a definition of the limits of the present invention, wherein:

FIG. 1 is a perspective view of a radiographic imaging apparatus according to an embodiment(s) of the present invention;

FIG. 2 is a block diagram showing electrical configuration of the radiographic imaging apparatus shown in FIG. 1;

FIG. 3 is a circuit diagram showing one of readout circuits of the radiographic imaging apparatus shown in FIG. 1;

FIG. 4 shows operation of the radiographic imaging apparatus shown in FIG. 1;

FIG. 5A is a graph of voltage(s) output from each of integrating circuits of the readout circuits shown in FIG. 3 in a case where switch elements are put into a conductive state in the middle;

FIG. 5B is a graph of voltage(s) output from the integrating circuits of the readout circuits shown in FIG. 3 in a case where the switch elements are not put into the conductive state (i.e. stay in a non-conductive state);

FIG. 6A is a graph where signal values read/obtained by conventional readout circuits with switch elements put in the non-conductive state, signal values of taken images, and differences between the signal values are plotted;

FIG. 6B is a graph where signal values read/obtained by the readout circuits shown in FIG. 3 with the switch elements put in the non-conductive state, signal values of taken images, and differences between the signal values are plotted;

FIG. 7A is a graph of voltage(s) output from each of integrating circuits of the conventional readout circuits;

FIG. 7B is an illustration to explain offset drift; and

FIG. 8 is an illustration to explain through current.

### DETAILED DESCRIPTION OF EMBODIMENTS

Hereinafter, one or more embodiment of the present invention will be described with reference to the drawings. However, the scope of the present invention is not limited to the disclosed embodiments.

Hereinafter, an indirect-type radiographic imaging apparatus that converts received radiation into light having a different wavelength, such as visible rays, with a scintillator will be described. The present invention is, however, also applicable to a direct-type radiographic imaging apparatus that directly detects radiation.

Furthermore, the radiographic imaging apparatus described hereinafter is portable. The present invention is, however, also applicable to an exclusive radiographic imaging apparatus installed/fixed, for example, in a room or on an imaging stand.

[Outline of Radiographic Imaging Apparatus]

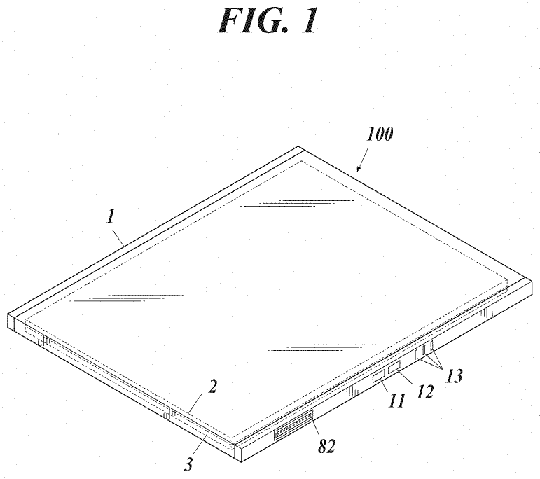

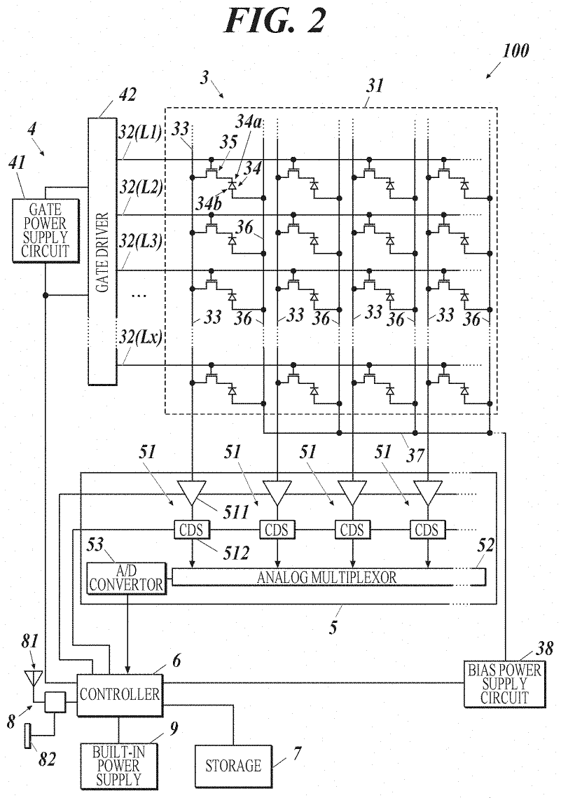

First, outline of a radiographic imaging apparatus (hereinafter “imaging apparatus 100”) according to an embodiment(s) will be described. FIG. 1 is a perspective view of the imaging apparatus 100 according to this embodiment. FIG. 2 is a block diagram showing electrical configuration of the imaging apparatus 100.

As shown in FIG. 1 and FIG. 2, the imaging apparatus 100 according to this embodiment includes: a case 1; and a scintillator 2, a radiation detector 3, a scan drive unit 4, a reader 5, a controller 6 (hardware processor), a storage 7, a communication unit 8, and a built-in power supply 9 that are housed in the case 1.

As shown in FIG. 1, the case 1 is panel-shaped and has approximately the same dimensions (size) as those of conventional medical radiation film cassettes.

As shown in FIG. 1, the case 1 has, on one lateral surface, a power switch 11, an operation switch 12, indicators 13, and a connector 82 of the communication unit 8, for example.

The scintillator 2 is made of a material that emits electromagnetic waves having a wavelength longer than that of radiation, such as visible rays, when receiving the radiation, and is plate-shaped.

The scintillator 2 may have a reflective layer on a surface facing the radiation detector 3 in order to transmit a larger amount of electromagnetic waves to the radiation detector 3.

The scintillator 2 may be formed of columnar crystals made of, for example, CsI (cesium iodide) in order to reduce diffusion of electromagnetic waves.

The radiation detector 3 includes, as shown in FIG. 2, a substrate 31, a plurality of scan lines 32, a plurality of signal lines 33, a plurality of radiation detection elements 34, a plurality of switch elements 35, a plurality of bias lines 36, a connection line(s) 37, and a bias power supply circuit 38.

The substrate 31 is plate-shaped, and provided parallel to the scintillator 2 so as to face the scintillator 2.

The scan lines 32 are provided parallel to one another at intervals of a predetermined distance.

The signal lines 33 are provided parallel to one another at intervals of a predetermined distance, and perpendicular and not electrically conductive to the scan lines 32.

That is, the scan lines 32 and the signal lines 33 are provided to be grid-like.

The radiation detection elements 34 are arranged so as to spread two-dimensionally on a surface of the substrate 31 and to face the scintillator 2. More specifically, the radiation detection elements 34 are provided in regions defined by the scan lines 32 and the signal lines 33 in the grid-like fashion on a surface of the substrate 31, thereby being arranged in a matrix.

The radiation detection elements 34 are configured to emit, with photodiodes, phototransistors, or the like, charges in amounts corresponding to doses of received radiation (or amounts of electromagnetic waves into which the scintillator 2 converts received radiation).

To one terminal 34a and the other terminal 34b of each radiation detection element 34, the drain of a switch element 35 and a bias line 36 are connected, respectively.

The switch elements 35 are provided in the regions defined by the scan lines 32 and the signal lines 33, as with the radiation detection elements 34.

To the gate, the source, and the drain of each switch element 35, a nearby scan line 32, a nearby signal line 33, and one terminal of the radiation detection element 34 in the same region are connected, respectively.

Each switch element 35 can switch a conductive state in which its corresponding radiation detection element 34 and signal line 33 (integrating circuit 511) are electrically conductive to one another and a non-conductive state in which its corresponding radiation detection element 34 and signal line 33 are not electrically conductive to one another.

The bias lines 36 are connected to the terminals 34b of the radiation detection elements 34.

In this embodiment, the bias lines 36 are tied by one connection line 37, but may be connected to the bias power supply circuit 38 directly, or tied by two or more connection lines 37. Tying the bias lines 36 with one connection line 37 concentrates current that flows in the bias lines 36, and makes a voltage drop due to wiring resistance large. Tying the bias lines 36 with two or more connection lines 37 divides the current, and makes the voltage drop small.

In order to reduce the influence of the wiring resistance, the bias line(s) 36 may be arranged in a planar shape on the whole surface of the substrate 31 or in sharp-signs shape, in which lines arranged vertically and horizontally are connected to one another at intersection points.

The bias power supply circuit 38 generates reverse bias voltage, and applies the reverse bias voltage to the radiation detection elements 34 via the bias lines 36.

The scan drive unit 4 includes a gate power supply circuit 41 and a gate driver 42.

The gate power supply circuit 41 generates ON voltage and OFF voltage having different voltages, and supplies the ON voltage and the OFF voltage to the gate driver 42.

The gate driver 42 can switch a voltage to be applied to each scan line 32 to the ON voltage or the OFF voltage.

The reader 5 includes a plurality of readout circuits 51, an analog multiplexor 52, and an A/D convertor 53.

Although not shown, the reader 5 includes a plurality of readout ICs 5A, and each readout IC 5A includes the plurality of readout circuits 51, the analog multiplexor 52, and the A/D convertor 53.

The readout circuits 51 are provided so as to correspond to the respective columns of the matrix in which the radiation detection elements 34 are arranged, and connected to the signal lines 33.

Each readout circuit 51 includes an integrating circuit 511 and a correlated double sampling circuit (hereinafter “CDS circuit 512”).

The readout circuits 51 generate analog signal values on the basis of the amounts of charges input from their corresponding signal lines 33, and output the analog signal values to the analog multiplexor(s) 52.

The readout circuits 51 will be detailed later.

To the analog multiplexor 52, output terminals of the readout circuits 51 are connected. The analog multiplexor 52 selectively switches the readout circuits 51 to be connected to the A/D convertor 53, thereby allowing signal values output from the readout circuits 51 to be input to the A/D convertor 53 one by one.

The analog multiplexor 52 may be configured to output the input signals (signal values) to the A/D convertor 53 two by two or more in order to generate each pixel from a plurality of pixels (e.g. generate each pixel by averaging four pixels).

The A/D convertor 53 successively converts the input analog signal values into digital signal values.

To the CDS circuits 512, A/D convertors 53 may be connected one-to-one. In this case, no analog multiplexor 52 is needed.

The controller 6 is constituted of: a computer including a CPU (Central Processing Unit), a ROM (Read Only Memory), a RAM (Random Access Memory), and an input-output interface that are connected to a bus; an FPGA (Field Programmable Gate Array); or the like (all not shown). The controller 6 may be constituted of an exclusive control circuit.

The CPU reads various programs stored in the storage 7 on the basis of control signals or the like from other apparatuses (radiation generator, console, etc.), loads the read programs into the RAM, and executes various processes in accordance with the loaded programs, thereby integrally controlling operations of the components of the imaging apparatus 100.

The storage 7 includes an SRAM (Static RAM), an SDRAM (Synchronous DRAM), an NAND flash memory, and/or an HDD (Hard Disk Drive).

The storage 7 stores, in addition to the various programs, parameters, files and so forth necessary for execution of the programs.

The communication unit 8 can communicate with other apparatuses (console, radiation generator, etc.) via an antenna 81 and/or the connector 82 wirelessly or with cables.

The built-in power supply 9 includes, for example, a lithium ion battery and/or a lithium ion capacitor, and supplies power to the components of the imaging apparatus 100.

[Readout Circuit]

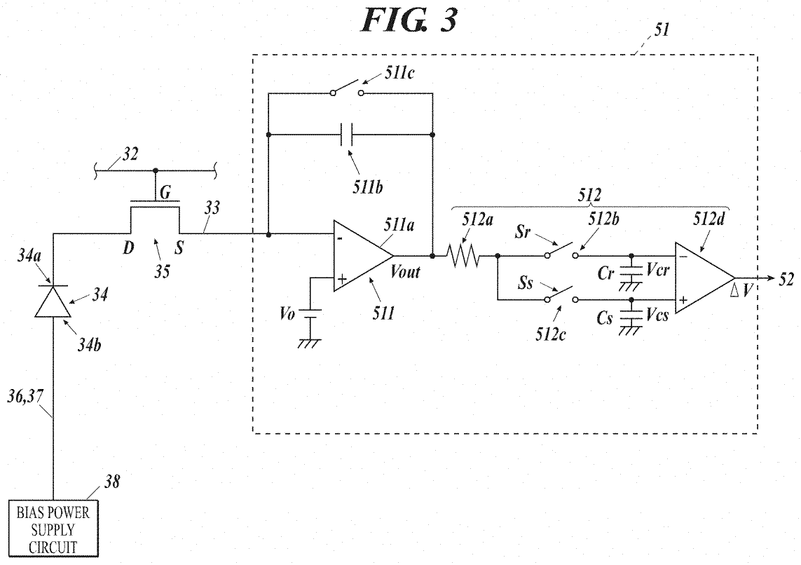

Next, the readout circuits 51 of the reader 5 of the imaging apparatus 100 will be detailed. FIG. 3 is a circuit diagram showing specific configuration of the readout circuit(s) 51.

As shown in FIG. 3, the readout circuit 51 includes the integrating circuit 511 and the CDS circuit 512.

The integrating circuit 511 includes an operational amplifier 511a, a capacitor 511b, and a reset switch 511c.

The operational amplifier 511a has an inverting input terminal to which its corresponding signal line 33 is connected and a non-inverting input terminal to which reference voltage V₀ is applied. Hence, a voltage applied to the signal line 33 is also the reference voltage V₀.

When charges flow into the operational amplifier 511a from the signal line 33, the operational amplifier 511a outputs an output voltage Vout corresponding to the amount of flowed-in charges.

The reference voltage Vo may be GND potential.

The capacitor 511b and the reset switch 511c are connected in parallel between the inverting input terminal and the output terminal of the operational amplifier 511a.

The integrating circuit 511 thus configured can output, to the CDS circuit 512, a voltage obtained by time integration of the amount of flowed-in charges.

When the reset switch 511c is put into the conductive state, charges accumulated in the capacitor 511b are released, and the voltage thereof is reset.

The CDS circuit 512 includes a resistor 512a, a first sample-and-hold circuit 512b, a second sample-and-hold circuit 512c, and a difference circuit 512d.

The resistor 512a is connected to the output terminal of the operational amplifier 511a of the integrating circuit 511 in series.

The first sample-and-hold circuit 512b includes a first capacitor Cr and a first switch Sr.

One electrode of the first capacitor Cr is connected between the resistor 512a and the inverting input terminal of the difference circuit 512d, and the other electrode of the first capacitor Cr is connected to GND.

The first switch Sr is provided between the resistor 512a and the first capacitor Cr.

When the first switch Sr is put into the conductive state, the integrating circuit 511 and the first capacitor Cr are connected to one another, so that the first capacitor Cr is charged.

Thereafter, when the first switch Sr is put into the non-conductive state, the integrating circuit 511 and the first capacitor Cr are disconnected from one another, and a voltage between the electrodes of the first capacitor Cr at this timing (hereinafter “first voltage Vcr”) is held.

The first sample-and-hold circuit 512b thus configured can sample the output (output voltage Vout) of the integrating circuit 511 and hold the sampled output as the first voltage Vcr.

The second sample-and-hold circuit 512c includes a second capacitor Cs and a second switch Ss.

One electrode of the second capacitor Cs is connected between the resistor 512a and the non-inverting input terminal of the difference circuit 512d, and the other electrode of the second capacitor Cs is connected to GND.

The second switch Ss is provided between the resistor 512a and the second capacitor Cs.

When the second switch Ss is put into the conductive state, the integrating circuit 511 and the second capacitor Cs are connected to one another, so that the second capacitor Cs is charged.

Thereafter, when the second switch Ss is put into the non-conductive state, the integrating circuit 511 and the second capacitor Cs are disconnected from one another, and a voltage between the electrodes of the second capacitor Cs at this timing (hereinafter “second voltage Vcs”) is held.

The second sample-and-hold circuit 512c thus configured can sample the output (output voltage Vout) of the integrating circuit 511 and hold the sampled output as the second voltage Vcs.

The first sample-and-hold circuit 512b and the second sample-and-hold circuit 512c are connected to the inverting input terminal and the non-inverting input terminal of the difference circuit 512d, respectively.

The difference circuit 512d outputs a difference as a signal value obtained by subtracting the first voltage Vcr held by the first sample-and-hold circuit 512b from the second voltage Vcs held by the second sample-and-hold circuit 512c.

This difference is output to the A/D convertor 53 via the analog multiplexor 52 as an analog signal value, described above, ΔV.

[Basic Operation]

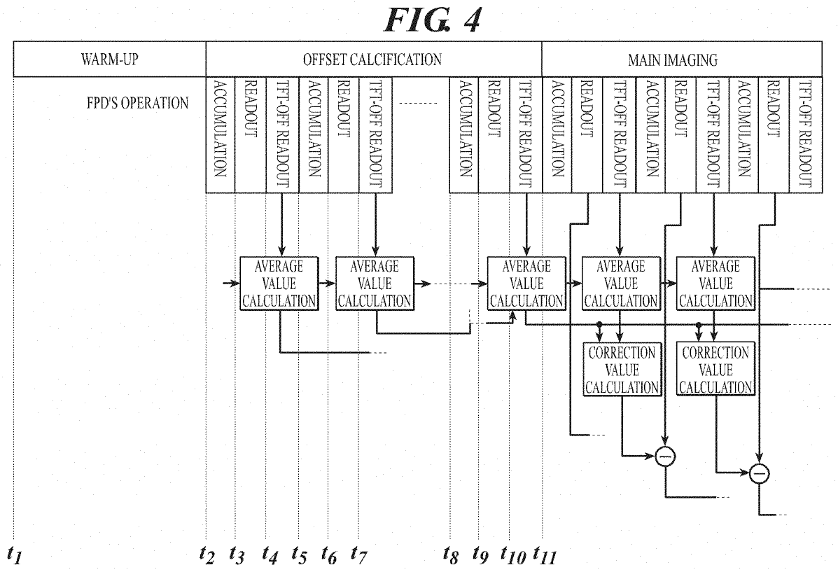

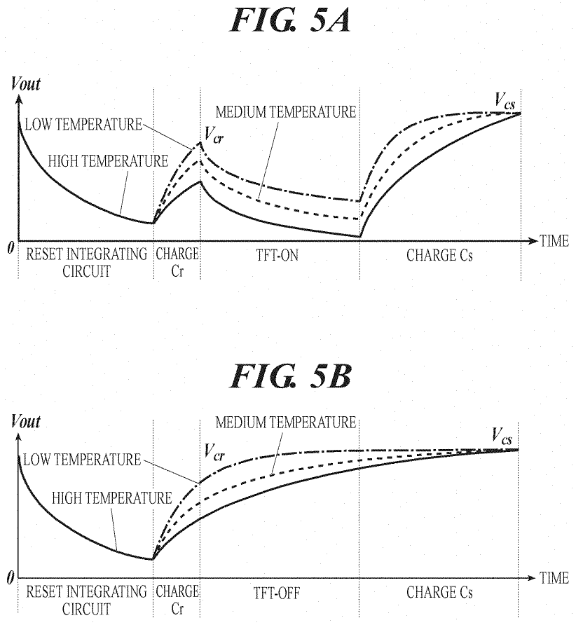

Next, operation of the imaging apparatus 100 will be described. FIG. 4 shows the operation of the imaging apparatus 100. FIG. 5A and FIG. 5B are graphs showing temporal change of the output voltage Vout of each integrating circuit 511.

The controller 6 of the imaging apparatus 100 causes the components of the imaging apparatus 100 to operate as described hereinafter.

When the power switch 11 is operated (to be ON), the controller 6 first causes the readout circuits 51 to apply the reference voltage Vo to the signal lines 33, and also causes the bias power supply circuit 38 to apply the reverse bias voltage to the radiation detection elements 34 via the connection line 37 and the bias lines 36.

Thereafter, as shown in FIG. 4, the controller 6 shifts the imaging apparatus 100 to a warm-up state (t1). In this state, the controller 6 causes the reader 5 to repeatedly perform a reading operation to increase temperature of the reader 5.

Increasing the temperature of the reader 5 to a certain level in this state slows down increase of the temperature of the reader 5 during video shooting.

In this embodiment, the controller 6 sets an imaging frame rate that is the number of radiographs generated per unit period of time, at any time before entering a main imaging state, which is described below.

The imaging frame rate may be set on the basis of an operation on the operation switch 12 or a numerical value obtained from another apparatus (e.g. console or radiation generator) connected to the imaging apparatus 100.

Thereafter, the controller 6 shifts the imaging apparatus 100 to an offset calibration state (t2). This state starts by taking, for example, any of the following timings in the warm-up state as an opportunity/trigger: when the reading operation has been performed a predetermined number of times; when a predetermined period of time has elapsed since start of the repeated reading operations; when the reader 5 has reached a predetermined temperature; and when a user performs a predetermined operation.

In this state, the controller 6 first causes the scan drive unit 4 to apply the OFF voltage to all the scan lines 32 to put all the switch elements 35 into the non-conductive state. Then, dark charges generated by the radiation detection elements 34 are accumulated in their corresponding pixels (t2 to t3).

Thereafter, the controller 6 causes the integrating circuits 511 to put the reset switches 511c into the conductive state to reset the integrating circuits 511. Then, as shown in FIG. 5A, the output voltages Vout of the integrating circuits 511 decrease.

When the integrating circuits 511 finish resetting their voltages, the controller 6 causes the first sample-and-hold circuits 512b to sample the output voltages Vout of their corresponding integrating circuits 511 for a predetermined period of time. More specifically, the controller 6 puts the first switches Sr into the conductive state to charge the first capacitors Cr.

In this embodiment, because the integrating circuits 511 output voltages (Vout) to their corresponding first sample-and-hold circuits 512b, the controller 6 causes the first sample-and-hold circuits 512b to sample the output voltages Vout, thereby holding the first voltages Vcr, simultaneously.

The sampled values are held (sample-held) as the first voltages Vcr.

Thereafter, the controller 6 causes the scan drive unit 4 to apply the ON voltage to one scan line 32 to put the switch elements 35 connected to the scan line 32 into the conductive state. Then, due to the voltage difference between the reference voltage Vo applied to the signal lines 33 and the reverse bias voltage applied to the bias lines 36, the dark charges accumulated in the pixels connected to the scan line 32 to which the ON voltage is applied are released to the signal lines 33 as dark current. Thus, the dark charges of one gate line are reset. At the time, due to through current, the increased output voltages Vout of the integrating circuits 511 decrease again.

When the dark current is released to the signal lines 33, the integrating circuits 511 of the readout circuits 51 each perform time integration of the dark current that flows in from their corresponding signal lines 33, and output the obtained output voltages Vout to their corresponding CDS circuits 512.

Thereafter, the controller 6 causes the second sample-and-hold circuits 512c to sample the output voltages Vout of their corresponding integrating circuits 511 for a predetermined period of time. More specifically, the controller 6 puts the second switches Ss into the conductive state to charge the second capacitors Cs.

In this embodiment, because the integrating circuits 511 output voltages (Vout) to their corresponding second sample-and-hold circuits 512c, the controller 6 causes the second sample-and-hold circuits 512c to sample the output voltages Vout, thereby holding the second voltages Vcs, simultaneously.

The sampled values are held (sample-held) as the second voltages Vcs.

As described above, in video shooting, due to short charging time of the second capacitors Cs and through current, output voltages (Vout) of the integrating circuits 511 are unlikely to be converged.

To deal with this, in this embodiment, the controller 6 causes the second sample-and-hold circuits 512c to sample the output voltages Vout for a period of time longer than the time constant τ of the second sample-and-hold circuits 512c (a product(s) of a resistance value(s) of the resistor(s) 512a and a capacitance(s) of the second capacitor(s) Cs).

It is preferable that the sampling time be within a range from three times to five times as long as the time constant τ.

This allows the second capacitors Cs to be charged until the output voltages Vout of the integrating circuits 511 are converged, so that the second voltages Vcs can be sampled accurately.

Although the sampling time of the second sample-and-hold circuits 512c can be extended, a period/cycle for each frame cannot be changed. Then, in this embodiment, the sampling time of the first sample-and-hold circuits 512b is changeable according to the set imaging frame rate. Hence, in the case of a large value of the set imaging frame rate, the sampling time of the first sample-and-hold circuits 512 is shortened by a period of time for which the sampling time of the second sample-and-hold circuits 512c is extended, so that the sampling time of the second sample-and-hold circuits 512c can be extended with the period/cycle for each frame unchanged.

Instead of the sampling time of the first sample-and-hold circuits 512b, time during which the integrating circuits 511 are reset or time during which the switch elements 35 are in the conductive state may be changed (shortened).

If the sampling time of the first sample-and-hold circuits 512b is shortened, the conductive state of the switch elements 35 starts before the output voltages Vout are converged, and the first voltages Vcr may not be sampled accurately. However, the second voltages Vcs can be sampled accurately at least, and hence the first voltages Vcr can be calculated on the basis of the second voltages Vcs accurately sampled/obtained by the second sample-and-hold circuits 512c and reference data obtained beforehand (e.g. obtained during the operation shown in FIG. 4 previously performed).

When the second sample-and-hold circuits 512c hold the second voltages Vcs, the difference circuits 512d output the analog signal values ΔV to the analog multiplexor(s) 52 as differences between the second voltages Vcs and the first voltages Vcr.

The analog multiplexor 52 successively outputs the input analog signal values ΔV to the A/D convertor 53.

Then, the A/D convertor 53 successively converts the input analog signal values ΔV into digital signal values.

The operations starting from application of the ON voltage to one scan line 32 up to this stage are repeated with the target scan line 32, to which the ON voltage is applied, being changed, so that image data of one frame is generated (t3 to t4).

The generated image data may be discarded without being used, or may be saved to be used, for example, in correction, which is described below.

After one frame is read, the controller 6 causes the scan drive unit 4 to apply the OFF voltage to all the scan lines 32 to put all the switch elements 35 into the non-conductive state (t4).

Then, in the non-conductive state, the controller 6 causes the first sample-and-hold circuits 512b to sample the output voltages Vout of the integrating circuits 511 for a predetermined period of time.

After the first sample-and-hold circuits 512b finish sampling the output voltages Vout, the controller 6 causes the second sample-and-hold circuits 512c to sample the output voltages Vout of the integrating circuits 511 for a predetermined period of time.

When the second sample-and-hold circuits 512c hold the second voltages Vcs, the difference circuits 512d output the analog signal values ΔV to the analog multiplexor(s) 52 as differences between the second voltages Vcs and the first voltages Vcr.

The analog multiplexor 52 successively outputs the input analog signal values ΔV to the A/D convertor 53.

Then, the A/D convertor 53 successively converts the input analog signal values ΔV into digital signal values.

Thus, TFT-OFF readout is performed (t4 to t5). In this sampling, which is performed while the switch elements 35 are in the non-conductive state, as shown in FIG. 5B, re-decrease of the output voltages Vout after resetting of the integrating circuits 511 does not occur. Hence, the first voltages Vcr and the second voltages Vcs can be sampled more accurately.

This sampling may be performed only one time or multiple times in one non-conductive state (i.e. one TFT-OFF readout).

During the offset calibration state, the controller 6 repeats this series of the operations, i.e. from accumulation of dark charges to TFT-OFF readout, multiple times (t5 to . . . , . . . , and t8 to t11).

That is, the controller 6 causes the first sample-and-hold circuits 512b to sample the output voltages Vout, thereby holding the first voltages Vcs, multiple times, and also causes the second sample-and-hold circuits 512c to sample the output voltages Vout, thereby holding the second voltages Vcs, multiple times.

The controller 6 obtains correction information based on the output of (i.e. differences output from) the CDS circuits 512 every predetermined number of times the sampling in the non-conductive state (TFT-OFF readout) is performed by the first sample-and-hold circuits 512b and the second sample-and-hold circuits 512.

In this embodiment, the controller 6 obtains correction information every time the sampling (TFT-OFF readout) is performed, but may obtain correction information once every N (an integer of two or more) times the sampling is performed.

In this embodiment, because the integrating circuits 511 output voltages (Vout) to their corresponding CDS circuits 512, the controller 6 obtains a plurality of pieces of correction information based on the output of (i.e. differences output from) the CDS circuits 512.

As described above, if the first sample-and-hold circuits 512b and the second sample-and-hold circuits 512c sample the output voltages Vout multiple times in one non-conductive state, the controller 6 may obtain correction information every time the CDS circuits 512 output the differences.

When obtaining correction information second time or thereafter, the controller 6 calculates, as the correction information, a moving average value of pieces of correction information that have been obtained so far (this time included).

The controller 6 uses, as reference offset data, a moving average value calculated at a late (preferably the last) time of the non-conductive state in the offset calibration state on the basis of pieces of correction information that have been obtained so far.

Thereafter, the controller 6 shifts the imaging apparatus 100 to the main imaging state (t11). This state starts by taking, for example, any of the following timings in the offset calibration state as an opportunity/trigger: when the reading operation has been performed a predetermined number of times; when a predetermined period of time has elapsed since start of the repeated reading operations; when a user performs a predetermined operation; and when radiation is detected.

The operation of the controller 6 in the main imaging state is basically the same as that in the offset calibration state except that in the main imaging state, the controller 6 calculates a correction value on the basis of correction information obtained.

In this embodiment, the controller 6 calculates a moving average value as correction information, and calculates a correction value by subtracting the reference offset data from the calculated moving average value.

The controller 6 may calculate, for each readout IC 5A, the correction value on the basis of the average value of pieces of correction information obtained from multiple pairs of the first sample-and-hold circuits 512b and the second sample-and-hold circuits 512c.

Alternatively, the controller 6 may calculate, for each pair of the first sample-and-hold circuit 512b and the second sample-and-hold circuit 512c (i.e. each CDS circuit 512), the correction value on the basis of the average value of pieces of correction information obtained from the pair multiple times.

Performing any of these can remove (cancel) noise components from the signal values read in the non-conductive state, and hence can improve correction accuracy.

Furthermore, the controller 6 may perform median filtering on the pieces of the correction information before calculating the correction value. This prevents decrease of correction accuracy due to abnormal signal values generated by breaking of the signal lines 33 or the like.

Two or more of the above processes may be performed in combination.

The controller 6 corrects, with the calculated correction value, the signal values (analog signal values or digital signal values) that are the output of (i.e. differences output from) the CDS circuits 512 after the second sample-and-hold circuits 512c sample the output voltages Vout via the conductive state.

In this embodiment, the controller 6 corrects the signal values with the correction value calculated at the timing (e.g. immediately before the correction) within a predetermined period of time from the timing at which the second sample-and-hold circuits 512c hold their respective second voltages Vcs.

Because the timing of obtaining/generating the correction value is before the timing of obtaining the frame to which the correction value is applied, the process flow can be simplified.

The controller 6 generates a radiograph(s) on the basis of the corrected signal values (analog signal values or digital signal values).

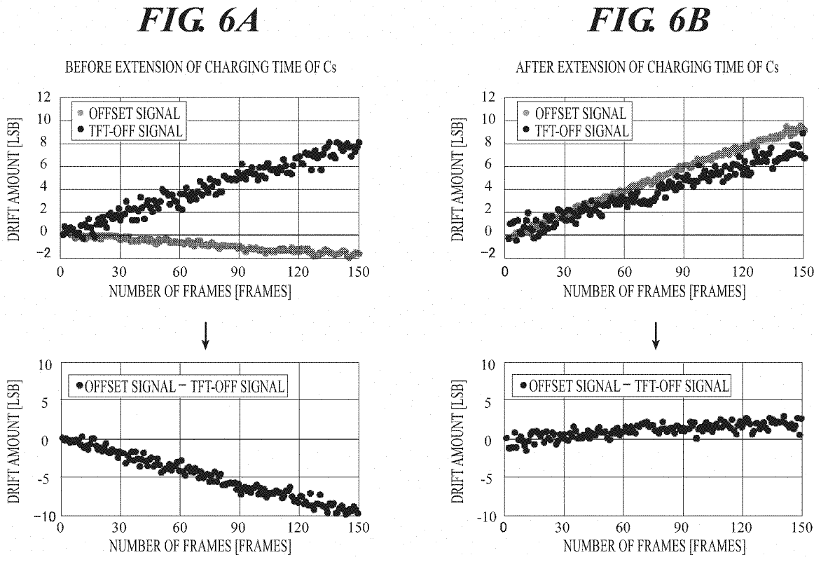

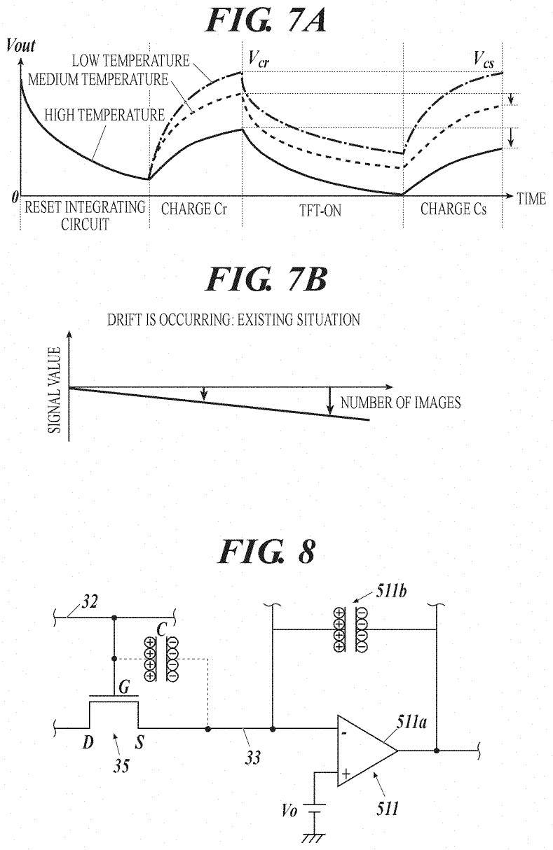

In a conventional radiographic imaging apparatus in which charging time of second capacitors Cs is short, as shown in FIG. 6A, as imaging progresses (as the number of frames increases), a gap between signal values read in the non-conductive state of switch elements (TFT-OFF signals) and signal values of taken images (offset signals) becomes bigger, and hence their differences (corrected signal values) cannot be uniform. (FIG. 6A shows a case where the corrected signal values decrease.)

Meanwhile, according to the imaging apparatus 100 of this embodiment, as shown in FIG. 6B for example, signal values read in the non-conductive state of the switch elements 35 (TFT-OFF signals) and signal values of taken images (offset signals) show almost the same increasing tendency, and hence their differences (corrected signal values) are around a certain value. That is, accuracy of offset correction in video shooting can be increased.

Needless to say, the present invention is not limited to the above embodiment and can be appropriately modified without departing from the scope of the present invention.

The information that the first voltages Vcr sampled in the non-conductive state have includes information on: noise generated when the integrating circuits 511 are reset; and degree of decrease of responsiveness of the CDS circuits 512, which is a factor of offset drift. Meanwhile, the information that the second voltages Vcs sampled in the non-conductive state includes only information on noise generated when the integrating circuits 511 are reset.

From the perspective of correction of offset drift, if the noise generated when the integrating circuits 511 are reset can be ignored, the second voltages Vcs being a fixed value have no problem. Hence, it is possible that as to the second voltages Vcs, a fixed value obtained beforehand is used, and as to the first voltages Vcr sampled in the non-conductive state, the noise that enters the signals values when the integrating circuits 511 are reset is ignored (e.g. cancelled by any of various averaging processes), and differences between these are output as the analog signal values ΔV.

Furthermore, although in the above description, a semiconductor memory and/or a hard disk are used as a computer-readable storage medium storing the program(s) disclosed herein, the computer-readable storage medium is not limited to these.

As the computer-readable storage medium, a nonvolatile memory, such as a flash memory, and a portable storage medium, such as a CD-ROM, may also be used.

Also, as a medium that provides, via a communication line, data of the program(s) disclosed herein, a carrier wave may be used.

Although some embodiments of the present invention have been described and shown in detail, the disclosed embodiments are made for purposes of illustration and example only and not limitation. The scope of the present invention should be interpreted by terms of the appended claims.

What is claimed is:

1. A radiographic imaging apparatus comprising:
a plurality of radiation detection elements that are arranged so as to spread two-dimensionally and generate charges in amounts corresponding to doses of received radiation;
an integrating circuit that outputs a voltage obtained by time integration of an amount of flowed-in charges;
a correlated double sampling circuit that: has (i) a first sample-and-hold circuit that samples the voltage output from the integrating circuit and holds the sampled voltage as a first voltage and (ii) a second sample-and-hold circuit that samples the voltage output from the integrating circuit and holds the sampled voltage as a second voltage; and outputs a difference between the second voltage and the first voltage;
a plurality of switch elements that switch a conductive state in which the corresponding radiation detection elements and the integrating circuit are electrically conductive to one another and a non-conductive state in which the corresponding radiation detection elements and the integrating circuit are not electrically conductive to one another; and
a hardware processor that:
causes the first sample-and-hold circuit to sample the voltage output from the integrating circuit for a predetermined period of time every time the integrating circuit finishes resetting a voltage;
causes the second sample-and-hold circuit to sample the voltage output from the integrating circuit for a period of time longer than time constant of the second sample-and-hold circuit after the first sample-and-hold circuit finishes the sampling;
obtains correction information based on the difference output from the correlated double sampling circuit every predetermined number of times the sampling in the non-conductive state is performed by the first sample-and-hold circuit and the second sample-and-hold circuit;
calculates a correction value based on the obtained correction information;
corrects, with the calculated correction value, a signal value that is the difference output from the correlated double sampling circuit after the second sample-and-hold circuit performs the sampling via the conductive state; and
generates a radiograph based on the corrected signal value.

2. The radiographic imaging apparatus according to claim 1, wherein the hardware processor:
sets an imaging frame rate that is the number of radiographs each being the radiograph generated per unit period of time; and
changes, according to the set imaging frame rate, the period of time for which the first sample-and-hold circuit performs the sampling.

3. The radiographic imaging apparatus according to claim 1, wherein
the radiation detection elements are arranged in a matrix,
the radiographic imaging apparatus comprises: a plurality of integrating circuits each being the integrating circuit; and a plurality of correlated double sampling circuits each being the correlated double sampling circuit, for respective columns of the matrix in which the radiation detection elements are arranged, and
the hardware processor:
causes the first sample-and-hold circuits to perform the sampling, thereby holding the respective first voltages, simultaneously;
causes the second sample-and-hold circuits to perform the sampling, thereby holding the respective second voltages, simultaneously;
obtains, as the correction information, a plurality of pieces of correction information based on the differences output from the respective correlated double sampling circuits; and
calculates the correction value based on an average value of the pieces of the correction information obtained from the respective correlated double sampling circuits.

4. The radiographic imaging apparatus according to claim 1, wherein the hardware processor:
causes the first sample-and-hold circuit to perform the sampling, thereby holding the first voltage, multiple times;
causes the second sample-and-hold circuit to perform the sampling, thereby holding the second voltage, multiple times;
obtains the correction information every time the correlated double sampling circuit outputs the difference, thereby obtaining a plurality of pieces of correction information; and
calculates the correction value based on an average value of the pieces of the correlation information.

5. The radiographic imaging apparatus according to claim 3, wherein the hardware processor performs median filtering on the pieces of the correction information before calculating the correction value.

6. The radiographic imaging apparatus according to claim 1, comprising a case having a size substantially identical with a size of a medical radiation film cassette, wherein
the radiographic imaging apparatus is portable.

7. The radiographic imaging apparatus according to claim 1, wherein the hardware processor corrects the signal value with the correction value calculated at a timing within a predetermined period of time from a timing at which the second sample-and-hold circuit holds the second voltage.

8. A non-transitory computer-readable storage medium storing a program to cause, of a radiographic imaging apparatus including: a plurality of radiation detection elements that are arranged so as to spread two-dimensionally and generate charges in amounts corresponding to doses of received radiation; an integrating circuit that outputs a voltage obtained by time integration of an amount of flowed-in charges; a correlated double sampling circuit that: has (i) a first sample-and-hold circuit that samples the voltage output from the integrating circuit and holds the sampled voltage as a first voltage and (ii) a second sample-and-hold circuit that samples the voltage output from the integrating circuit and holds the sampled voltage as a second voltage; and outputs a difference between the second voltage and the first voltage; and a plurality of switch elements that switch a conductive state in which the corresponding radiation detection elements and the integrating circuit are electrically conductive to one another and a non-conductive state in which the corresponding radiation detection elements and the integrating circuit are not electrically conductive to one another, a computer to:
cause the first sample-and-hold circuit to sample the voltage output from the integrating circuit for a predetermined period of time every time the integrating circuit finishes resetting a voltage;
cause the second sample-and-hold circuit to sample the voltage output from the integrating circuit for a period of time longer than time constant of the second sample-and-hold circuit after the first sample-and-hold circuit finishes the sampling;
obtain correction information based on the difference output from the correlated double sampling circuit every predetermined number of times the sampling in the non-conductive state is performed by the first sample-and-hold circuit and the second sample-and-hold circuit;
calculate a correction value based on the obtained correction information;
correct, with the calculated correction value, a signal value that is the difference output from the correlated double sampling circuit after the second sample-and-hold circuit performs the sampling via the conductive state; and
generate a radiograph based on the corrected signal value.
